(19) United States
(12) Patent Application Publication
(10) Pub. No.: US 2016/0000699 A1
(43) Pub. Date: Jan. 7, 2016

(54) HAIR GROWTH PROMOTING CAPACITY OF CONDITIONED MEDIA OF STIMULATED STEM CELLS AND USE THEREOF

(71) Applicant: MEDIPOST Co., Ltd., Seoul (KR)

(72) Inventors: Yoon Sun YANG, Seoul (KR); Wonil OH, Seoul (KR); Jang Young LEE, Seoul (KR); Soo Jin CHOI, Seoul (KR); Hong Bae JEON, Seoul (KR); Ju-Yeon KIM, Seoul (KR); Hoon LIM, Seoul (KR); Seung-Hyun SHIN, Yongin-si (KR); Won-Seok PARK, Yongin-si (KR)

(73) Assignee: MEDIPOST Co., Ltd., Seoul (KR)

(21) Appl. No.: 14/792,368

(22) Filed: Jul. 6, 2015

Related U.S. Application Data
(60) Provisional application No. 62/021,366, filed on Jul. 7, 2014.

(51) Int. Cl. A61K 8/98; A61Q 7/00

(52) U.S. Cl. CPC A61K 8/981; A61Q 7/00; A61K 2800/92; A61K 2800/91

(57) ABSTRACT

Provided is an a composition for preventing hair-loss and promoting hair growth containing as an active ingredient conditioned media of stem cells stimulated by particular hair catagen inducers including TGF-β, a method of manufacturing the same, and a use thereof. In particular, the present invention relates to a use of the function of very effective hair growth by secreting Wnt3a, Bcl-2, CyclinD-1 and the like, which are known as signal transduction proteins.

### CROSS-REFERENCE TO RELALED PATENT APPLICATION

This application claims priority to and the benefit of U.S. Patent Application No. 62/021,366 filed on Jul. 7, 2014 the disclosure of which is incorporated herein by reference in its entirety.

### BACKGROUND OF THE INVENTION

1. Field of the Invention

The present invention relates to a composition for preventing hair-loss and promoting hair growth containing, as an active ingredient, a conditioned medium of stem cells stimulated by a particular catagen inducer including TGF-β, a method of manufacturing the same, and a use thereof.

2. Description of the Related Art

With the progress of industrial development, environmental pollution, stress, and aging, the symptoms of alopecia or hair-loss symptoms become more serious, and with the advent of well-being era, there are growing concerns on the quality of life and physical appearance. Alopecia, a symptom of hair-loss from scalp, is caused by various reasons including, for example, intrinsic factors such as genetic traits and the actions of male hormones; mental stress in daily life; and extrinsic factors such as accumulation of lipid peroxide, and the alopecia symptoms are known to be caused by a very complex process.

Hair baldness does not mean that hair is lost and never grows again, but the hair progressively becomes thinner into a downy hair, and dermal papilla present in hair root becomes small. As the dermal papilla becomes smaller, hair thickness also becomes thinner and hair cycle becomes shorter thereby making the newly growing hair thinner. As baldness progresses continuously, hairs become downy hairs and make hair cycle shorter thereby losing hair after short growth. Additionally, alopecia areata, known to be an autoimmune disease, and temporary alopecia occurring due to endocrine disease, nutrient deficiency, drugs, physical or mental stress such as delivery, etc., have been also concerned.

Recently, not only male-type alopecia but also obese alopecia of females, and young adult alopecia are gradually on the increase. According to a release in 2009 by the Institute for Health and Welfare Policy of National Insurance Service in Korea, the number of domestic alopecia patients in 2008 was increased at least 60%, compared with that of 2001. In addition, and the number of children and adolescent alopecia patients increased from 21,643 in 2006 to 23,025 in 2011, showing about 6.4% increase in 5 years.

In order to remedy the hair-loss phenomena described above, many types of hair-growth agents and hair tonics have been available on the market. According to a report by Economy21, in the current domestic market of hair-loss services in Korea, cosmetics and quasi-drugs account for 80%, and pharmaceutical drugs account for about 20%, and among them, alopecia patients who see doctors account for only 5%. At present, among the people who use hair growth related products, about 72.7% of the users are not satisfied with the products, and there are no proven therapies for hair-loss treatment, except the two US FDA approved preparations of Minoxidil® and Finasteride® released in 1998, i.e., about 15 years ago. Accordingly, the two preparations are not sufficient to cure all the various hair-loss related symptoms. In particular, Propecia®, which is prepared as a finasteride preparation, is not an agent for hair growth but a hair-loss preventing agent. Propecia® only has an effect of maximally delaying the progress of a male-pattern alopecia via inhibition of DHT production by blocking 5-α reductase.

In the case of hair-growth agents and hair tonics currently available on the market (e.g., Minoxidil® and Finasteride®), the agents may cause side effects due to application of hormonal preparations or they exhibit effects only in the areas where the hair roots are activated. Accordingly, although the agents have effects regarding the prevention of hair-loss, their effects on the growth of hairs in a long-term telogen phase (i.e., hair growth) are either negligible, or they may exhibit effects only when they are continuously administered or applied to, and thus it is necessary to develop a cost-effective and stable technology for resolving alopecia or hair-loss symptoms. The level of research and development of therapeutic agents for alopecia in Korea is technologically behind that of advanced countries, and there is an urgent need for the research and development of therapeutic agents for prevention of hair-loss, promotion of hair-growth, and hair-rejuvenation. As for the research on hair-loss in Korea, reports on hair growth and rejuvenation of hair root have been published since 1950, but hair rejuvenation had been considered as impossible due to lack of reproducibility, etc., for the last five decades.

However, professor Costarelis at School of Medicine, University of Pennsylvania, USA, firstly discovered hair follicle stem cells in 1990 (Cell, 1990), succeeded in separating human follicle stem cells (J. Clin. Invest, 2006), and released his research results on possibility of hair follicle regeneration (Nature, 2007), the reproducibility of which had been considered impossible by then, and opened a possibility of researching the fundamental treatment of baldness itself.

Recently, methods of treating hair-loss using genes and stem cells have been developed. As a basis of the present invention, Korean Patent No. 10-0771171 (Oct. 29, 2007) describes a method for isolation, expansion and differentiation of a hair follicle stem cell, and a therapeutic composition for baldness. Korean Patent Application Publication No. 10-2008-0097593 in (Nov. 6, 2008) describes a cellular therapeutic agent comprising multipotent stem cells derived from human adipose tissue and hair follicle cells. Additionally, Korean Patent No. 10-1218101 (Jan. 3, 2013) describes a composition for promoting hair growth and preventing hair-loss comprising a fetus-derived mesenchymal stem cell from amniotic fluid as an active ingredient.

However, the effects of the above agents using the stem cells on hair-growth have not been sufficient, and thus various attempts using stem cells are being made to develop effective therapeutic agents for treating hair-loss.

In this regard, the present inventors, while endeavoring to identify the effects of mesenchymal stem cells derived from umbilical cord blood on hair-growth, discovered that the treatment of TGF-β, which is known as a material causing hair-loss, on umbilical cord blood stem cells, contrary to the previous notion, secretes proteins effective on hair-growth, and also confirmed that the conditioned medium obtained by culturing the stem cells stimulated with TGF-β exhibited far superior hair-growth promoting effect than that of the previously known cases, thereby completing the present invention.

### PRIOR ART DOCUMENTS

### Patent Documents

1. Korean Patent No. 10-0771171 (Oct. 29, 2007)
2. Korean Patent No. 10-1422559 (Jul. 17, 2014)
3. Korean Patent Application Publication No. 10-2013-0009117 (Jan. 23, 2013)
4. Korean Patent Application Publication No. 10-2014-0125735 (Oct. 29, 2014)
5. Korean Patent Application Publication No. 10-2008-0097593 (Nov. 6, 2008)
6. Korean Patent No. 10-1218101 (Jan. 3, 2013)

### Non-Patent Documents

1. Dong L, Hao H, Xia L, Liu J, Ti D, Tong C, Hou Q, Han Q, Zhao Y, Liu H, Fu X, Han W; Treatment of MSCs with Wnt1a-conditioned medium activates DP cells and promotes hair follicle regrowth.; Sci Rep. 2014 Jun. 25; 4:5432. doi: 10.1038/srep05432.
2. Park B S, Kim W S, Choi J S, Kim H K, Won J H, Ohkubo F, Fukuoka H.; Hair growth stimulated by conditioned medium of adipose-derived stem cells is enhanced by hypoxia: evidence of increased growth factor secretion.; Biomed Res. 2010 February; 31(1):27-34.
3. Jeong Y M, Sung Y K, Kim W K, Kim J H, Kwack M H, Yoon I, Kim D D, Sung J H.; Ultraviolet B preconditioning enhances the hair growth-promoting effects of adipose-derived stem cells via generation of reactive oxygen species.; Stem Cells Dev. 2013 Jan. 1; 22(1):158-68. doi: 10.1089/scd.2012.0167. Epub 2012 Aug. 13.

Over the entire specification of the present invention, many references and patent documents are referred to and their citations are indicated thereon as appropriate. The contents of disclosure of the cited references and patent documents are incorporated in their entireties as references into the specification, and thereby, the level of technical field to which the present invention pertains and the contents of the disclosure of the present invention are explained more clearly.

### SUMMARY OF THE INVENTION

The present invention employs a conditioned medium of stem cells stimulated by a particular hair catagen inducer, which secretes signal transduction proteins associated with hair tissue differentiation by stimulating stem cells, and a main object of the present invention is to provide a composition for preventing hair-loss and promoting hair-growth containing the conditioned medium of stem cells stimulated by a particular hair catagen inducer.

Another object of the present invention is to provide a method for preventing hair-loss and promoting hair-growth using the composition.

A further object of the present invention is to provide a method for effectively using the composition.

In order to achieve the above objects, the present invention provides various uses of employing very effective hair-growth functions via secretion of signal transduction proteins associated with hair tissue differentiation by stimulating stem cells with particular hair catagen inducers including TGF-β.

In an exemplary embodiment, the present invention provides a composition for preventing hair-loss and promoting hair growth comprising as an active ingredient conditioned medium of stem cells stimulated by at least one hair catagen inducer selected from the group consisting of TGF-β, IFN-γ, FGF-5, IL-1β, TNF-α, K17, NT-3, NT-4, BDNF, and BMP2/4. Preferably, the present invention provides a conditioned medium stimulated by at least one factor including TGF-β.

The effects of preventing hair-loss and promoting hair-growth are achieved because the stem cells stimulated by the hair catagen inducer are rendered the effects of secreting Wnt3a, Bcl-2, CyclinD-1, etc., which are known as signal transduction proteins associated with hair tissue differentiation.

More specifically, the condition medium, stimulated by at least one hair catagen inducer selected from the group consisting of TGF-β, IFN-γ, FGF-5, IL-1β, TNF-α, K17, NT-3, NT-4, BDNF, and BMP2/4, possesses the following functions of:

(i) reducing the conversion time from telogen phase to anagen phase during hair cycle;

(ii) producing dermal papilla cells and promoting the growth in length;

(iii) increasing the number and size of follicles; and

(iv) increasing the thickness of scalp skin.

In particular, the stem cell may be at least one selected from the group consisting of human tissue adult stem cells and embryonic stem cells derived from bone marrow, umbilical cord blood, adipose, blood, liver and intestines, skin, gastrointestinal tract, placenta, nerves, adrenal gland, epithelium, and uterus. Preferably, the stem cell may be derived from bone marrow, umbilical cord blood, or adipose; and more preferably, an adult stem cell derived from umbilical cord blood, e.g., a mesenchymal cell derived from umbilical cord blood. Furthermore, it is preferable that the umbilical cord blood to be used is one derived from humans.

The conditioned medium of stem cells stimulated by the hair catagen inducer is preferably at a final concentration of 10% to 30%, and more preferably a final concentration of 25%.

Additionally, the conditioned medium to be used may be any basic culture medium suitable for animal cell growth, and non-limiting examples of the medium may include Minimal Essential Medium (MEM), Dulbecco modified Eagle Medium (DMEM), Roswell Park Memorial Institute Medium (RPMI), and Keratinocyte Medium (KM), KBM (Keratinocyte Basal medium), EpiLife KM (Keratinocyte-EpiLife medium) etc. and preferably, KM (Keratinocyte medium), KBM (Keratinocyte Basal medium), EpiLife KM (Keratinocyte-EpiLife medium).

In particular, the conditioned medium of stem cells stimulated by the hair catagen inducer selected from the group consisting of TGF-β, IFN-γ, FGF-5, IL-1β, TNF-α, K17, NT-3, NT-4, BDNF, BMP2/4, etc., may be obtained by stimulating the stem cells for 22 to 26 hours after adding the hair catagen inducer to the stem cells, followed by culturing for a selected period of time of 1 to 3 days. More specific examples may be referred to Examples of the present invention.

In the present invention, the stem cells stimulated by the hair catagen inducer including TGF-β secrete Wnt3a, Bcl-2, CyclinD-1, etc., which are known as the signal transduction proteins associated with hair tissue differentiation, while the culture of the stem cells, the conditioned medium of stem cells of the present invention are characterized in that the conditioned medium includes at least one protein selected from the group consisting of Wnt3a, Bcl-2, and CyclinD-1.

The composition of the present invention may be prepared and provided in the form of a pharmaceutical composition or a cosmetic composition.

Meanwhile, in another exemplary embodiment, the present invention may provide a preferred method of using the composition for preventing hair-loss and promoting hair-growth containing the conditioned medium of stem cells stimulated by the hair catagen inducer selected from the group consisting of TGF-β, IFN-γ, FGF-5, IL-1β, TNF-α, K17, NT-3, NT-4, BDNF, and BMP2/4, explained above.

Additionally, the present invention may provide a method of treating hair-loss via promotion of hair-growth, which employs the hair-growth promoting composition containing the conditioned medium of stem cells stimulated by the hair catagen inducer as an active ingredient.

In the methods of the present invention, preferably, topical spreading or an injection may be used, and more preferably, topical spreading.

As such, the present invention, being based on the discovery of the fact that the treatment of hair catagen inducers such as TGF-β, BMPa, etc., which are known as hair-loss inducing materials, on stem cells derived from umbilical cord blood could secrete proteins effective on hair-growth, contrary to the conventional notion, provides the excellent hair-loss preventing and hair-growth effects of the conditioned medium of stimulated stem cells, and various uses of the same.

### BRIEF DESCRIPTION OF THE DRAWINGS

The patent or application file contains at least one color drawing. Copies of this patent or patent application publication with color drawing will be provided by the USPTO upon request and payment of the necessary fee.

The above and other features and advantages of the present invention will become more apparent by describing in detail exemplary embodiments thereof with reference to the attached drawings in which:

FIG. 1 is a graph showing the evaluation results of cellular proliferation of hDPCs treated with conditioned media of stem cells derived from various sources, stimulated by TGF-β and a control group;

FIG. 2 is a western blot result confirming the expression level of signal transduction system-related proteins associated with hair tissue differentiation, which are contained in the conditioned media of stem cells stimulated by TGF-β;

FIG. 3 is a result confirming the growth in length of dermal papilla (DP) cells, after treating with conditioned media of stem cells stimulated by TGF-β and a control group;

FIG. 4 is a graph and a stereomicroscope picture regarding the number of follicle formation, after treating with the conditioned media of stem cells stimulated by TGF-β and a control group; and

FIG. 5 is a picture confirming the hair growth feature, after coating CH3 mice for 3 weeks with the conditioned media of stem cells derived from UCB, stimulated by TGF-β, and a control group.

FIG. 6 is a picture confirming the hair growth feature, after coating CH3 mice for 4 weeks (28 days) with the conditioned media of stem cells derived from adipose and bone marrow, stimulated by TGF-β, and a control group.

### DETAILED DESCRIPTION OF THE INVENTION

Hereinafter, preferred embodiments will be described in more detail with reference to the accompanying drawings. Moreover, detailed descriptions of well-known functions or configurations will be omitted in order not to unnecessarily obscure the focus of the present invention.

The terms used in the present invention may be defined as described herein below.

“Hair-growth promotion” and “hair-loss prevention” are terms having similar meanings, and they refer to all the effects of promoting hair formation and hair-growth, and preventing hair-loss or hair-weakening in the art.

As used herein, the term “stem cell” refers to a cell which can be developed into any tissue. The two basic features of a stem cell are self-renewal capable of producing selves via repeated divisions, and multi-differentiation potential capable of differentiation into cells with a particular function depending on the environments.

As used herein, the term “mesenchymal stem cell” refers to a kind of undifferentiated adult stem cell isolated from humans or mammals, and may be derived from various tissues. Hematopoietic stem cells, among the adult stem cells, are mainly present in a non-adherent state but the mesenchymal stem cells are usually adherent cells. In particular, the mesenchymal stem cell may be an umbilical cord-derived mesenchymal stem cell, an umbilical cord blood-derived mesenchymal stem cell, a bone marrow-derived mesenchymal stem cell, a adipose-derived mesenchymal stem cell, a muscle-derived mesenchymal stem cell, a neuron-derived mesenchymal stem cell, a skin-derived mesenchymal stem cell, an amniotic membrane-derived mesenchymal stem cell, and a placenta-derived mesenchymal stem cell, and preferably, an umbilical cord blood-derived mesenchymal stem cell. The technologies for isolating stem cells from each tissue are already known in the art.

As used herein, the term “conditioned medium” refers to a material including constitutional components contained in a medium obtained by culturing stem cells, and the stem cell for preparing the above conditioned medium is not limited with regard to its kind. For example, the stem cell for preparing the conditioned medium may be an embryonic stem cell or an adult stem cell. Furthermore, the adult stem cell may be derived from the adult stem cells of all tissues. In an exemplary embodiment of the present invention, the conditioned medium was prepared using the umbilical cord blood-derived adult stem cell. Preferably, the stem cell was obtained via stimulation by adding TGF-β thereto for 22 to 26 hours, followed by culturing for a selected period time of 1 to 3 days.

As used herein, the term “differentiation” refers to a phenomenon in a cell where a structure or function of the cell is specialized during the cell growth via cell division, that is, the progressive changes in shape or function of a cell or tissue of an organism so that they can perform the work assigned thereto. Generally, it refers to a phenomenon of division of a relatively simple system into two or more of partial systems of different quality.

As used herein, the term “proliferation” or “growth” of a cell refers to an increase of a cell with the same quality via cell division, and usually refers to an increase in the number of cells in the body of a multi-cellular organism. When the number of cells reaches a certain period after proliferation (amplification) the trait of the cell becomes regulated while it is being changed (differentiated).

As used herein, the term “medium” refers to a mixture necessary for in vitro growth and proliferation of a cell including elements essential for cell growth and proliferation, such as amino acids, various nutrient materials, blood serum, growth factors, inorganic materials, etc. In particular, the medium of the present invention is a medium for growth and proliferation of a stem cell.

As used herein, the term “basal medium” refers to a mixture containing saccharides, amino acids, water, etc., required for the survival of a cell, exclusive of blood serum, nutrient materials, and various growth factors. The basal medium of the present invention may be used after preparation via artificial synthesis, or by purchasing the commercially available media. The commercially prepared media may include, for example, Dulbecco's Modified Eagle's Medium (DMEM), Endothelial differentiation medium (EDM), Minimal Essential Medium (MEM), Basal Medium Eagle (BME), RPMI 1640, F-10, F-12, α-Minimal Essential Medium (α-MEM), Glasgow's Minimal Essential Medium (G-MEM), and Iscove's Modified Dulbecco's Medium, but are not limited thereto.

As used herein, the term “treatment” refers to an access for the purpose of obtaining an advantageous or preferable clinical result. For the benefits of the present invention, the advantageous or preferable clinical result may include, as non-limiting examples, alleviation in symptoms, decrease in the scope of diseases, stabilization of a disease state (i.e., not being worsened), delay or decrease in progress of a disease, a (partial or overall) improvement or temporary alleviation and amelioration in a disease state, and presence of being detectable or undetectable. The term “treatment” refers to both therapeutic treatment and preventative or preventative treatment method. The treatments include not only the preventable disablement but also the treatment required in the disablement occurred already. The term “palliating” refers to, as compared to the case without treatment, a reduction in the range of a disease state and/or undesirable clinical signs, and/or prolonging or extending the time course of the progress.

As used herein, the term “effective amount” refers to a suitable amount that can affect on the result of an advantageous or desirable clinical or biochemical result. The effective amount may be administered once or more. The effective amount is an amount suitable for temporarily alleviating, improving, stabilizing, reversing, slowing down, or delaying the progress of a disease state. In the present invention, the effective amount refers to the amount required to alleviate or delay of the progress of hair-loss, or to promote hair-growth. If an animal to receive benefit can tolerate the administration of a composition or the administration is suitable for the animal, the composition is considered as “pharmaceutically or physiologically acceptable”. When the administered amount is physiologically important the preparation may be considered as having been administered in an “therapeutically effective amount”. When the presence of the preparation itself causes a physiologically detectable change in the number of beneficial patients the preparation is considered as physiologically meaningful.

As used herein, the term “about” refers to an amount, a level, a value, a number, a frequency, a percent, a dimension, a size, an amount, weight, or length, at the extent of a change of 30, 25, 20, 25, 10, 9, 8, 7, 6, 5, 4, 3, 2, or 1%, with regard to the reference the amount, the level, the value, the number, the frequency, the percent, the dimension, the size, the amount, the weight, or the length.

In the specification of the present invention, unless otherwise necessary in the context, the terms “include” and “including” should be interpreted as including the suggested steps or components, or a group of the steps or components, but not exclusive of other optional steps or components or a group of the steps or components.

The present invention will be described in details herein below.

The present invention relates to a particular function of a conditioned medium of stem cells treated with particular factors, that is, the functions of preventing hair-loss and promoting hair-growth, and uses thereof.

The factors that may be used as a hair catagen inducer for the significant prevention of hair-loss and promotion of hair-growth are at least one selected from the group consisting of TGF-β, FGF-5, IL-1β, TNF-α, K17, NT-3, NT-4, BDNF, BMP2/4, etc., and most preferably, one including TGF-β.

A stem cell is a cell capable of self-replication and differentiation into two or more cells, and can use an embryonic stem cell or an adult stem cell depending on the source it is derived from. In the present invention, the stem cell to be used may be an adult stem cell derived from various tissues of origin, for example, tissues derived from adipose, uterus, bone marrow, muscle, placenta, umbilical cord blood, or skin (epithelium). In particular, mesenchymal stem cells (MSCs) are preferred. The mesenchymal stem cells are generally stroma which help hematopoiesis, and can be differentiated into various mesodermal cells, and easily proliferate while maintaining undifferentiated state. In an exemplary embodiment, mesenchymal stem cells (MSCs) derived from adipose, bone marrow, and umbilical cord blood were used. Most preferably, the mesenchymal stem cells derived from umbilical cord blood are used.

Umbilical cord blood refers to a blood that comes from the umbilical cord, and contains a large amount of hematopoietic stem cells, which produce white blood cells, red blood cells, platelets, etc., and endothelial progenitor cells, and also contains mesenchymal stem cells, which produce cartilages, bones, muscles, nerves, etc., thus having a high medicinal value. The characteristics of umbilical cord blood are that not only the number of hematopoietic stem cells is present at a higher concentration in umbilical cord blood than that of the bone marrow or peripheral blood, but also that umbilical cord blood has significantly higher capabilities of proliferation, self-replication, and differentiation than the hematopoietic stem cells which are found in bone marrow. Additionally, the umbilical cord blood can be obtained from umbilical cord to be discarded via a simple surgical procedure, and contains a relatively large amount of hematopoietic stem cells and stem cells for its amount. Accordingly, in a preferred embodiment, the present invention uses human umbilical cord blood-derived mesenchymal stem cells (hUCB-MSC).

In particular, the umbilical cord blood-derived mesenchymal stem cells (i) when used as a cell therapeutic agent, have almost no immunological rejection unlike stem cells derived from other tissues, (ii) accompany no pains of the subject from which the stem cells are obtained because they are collected from placenta and umbilical cord to be discarded, and (iii) when applied, a direct application into the area with a disease is possible. In particular, it has an advantage in that when it is transplanted into the real target area, a paracrine effect becomes activated, and thus secretome factors (proteins, cytokines), which are capable of treating, renewing, or recovering the area with the disease, thus curing the disease.

The method to be used for isolating and culturing the mesenchymal stem cells collected from the umbilical cord blood may be any method known in the art (Pittinger M F, Mackay A M, et al., Science, 284:143-7, 1999; Lazarus H M, Haynesworth S E, et al., Bone Marrow Transplant, 16:557-64, 1995), for example, all the conventional methods including the method disclosed in Korean Patent No. 10-0494265 may be used. In an embodiment of the present invention, the following method may be used.

Mononuclear cells are separated from the collected umbilical cord blood by centrifugation, and washed a few times to remove impurities. The resulting mononuclear cells are cultured by planting them in a culture container at an appropriate density, and the cells are allowed to proliferate while forming a single layer. Here, the cells, when observed under a phase contrast microscope, proliferating in the form of a colony with a long homogeneous spindle shape are the mesenchymal stem cells. When the cells are grown to be confluent the cells are then subcultured and proliferated until an appropriate number of cells are obtained.

In particular, the present invention is characterized in that the stem cells are preferably stimulated by treating with a particular hair catagen inducer. The catagen inducer includes at least one factor selected from the group consisting of TGF-β, IFN-γ, FGF-5, IL-1β, TNF-α, K17, NT-3, NT-4, BDNF, and BMP2/4, and most preferably TGF-β.

The concentration of the hair catagen inducer for treatment is at 8-15 ng/mL relative to the cell volume, preferably 8-12 ng/mL, and most preferably 9-11 ng/mL. In an exemplary embodiment of the present invention, the cells were treated with the hair catagen inducer at about 10 ng/mL when the cell confluence was determined to be 80% or higher.

The TGF-β is a cytokine with various functions, and is a material known to be closely associated with cell growth and differentiation, inflammatory responses, cell apoptosis, and matrix synthesis of cells, by regulation of the expression of TGF-β related genes via Smads, which is a transcription factor present in cytoplasm, and has been reported to be involved in necrosis of follicle cells.

The biggest cause of hair-loss is that dihydrotestosterone (DHT), a hormone, is conjugated to testosterone and 5-α-reductase, a reducing agent, and the DHT entered into a normal hair cell delivers a DNA cell destroying signal of a nucleus thus leading to hair-loss. In particular, the follicle cell apoptosis factor attacks the neighboring follicle cells by the DNA destroying signal of a hair cell thereby leading to hair-loss, and examples of the necrosis factors of follicles include BMP, DKK-1, and TGF-β (J. Cell. Sci 2006, J. Anat. 2003). That is, the TGF-β or BMP or the like are materials known to induce hair-loss.

Therefore, treating stem cells with these materials for the effects of preventing hair-loss and promoting hair-growth are technologies not to be easily conceived by one of ordinary skill in the art, and it is also a feature that constitutes an important characteristic of the present invention.

The present inventors firstly confirmed that stimulating stem cells by treating with at least one hair catagen inducer selected from the group consisting of TGF-β, IFN-γ, FGF-5, IL-1β, TNF-α, K17, NT-3, NT-4, BDNF, and BMP2/4 secrete materials effective for hair-growth.

The stem cells stimulated with these factors secrete Wnt3a, Bcl-2, CyclinD-1, etc., which are known as signal transduction proteins associated with hair tissue differentiation, and the functions of preventing hair-loss and promoting hair-growth can be further improved by these proteins.

Accordingly, in the present invention, the conditioned media of stem cells stimulated by at least one hair catagen inducer selected from the group consisting of TGF-β, IFN-γ, FGF-5, IL-1β, TNF-α, K17, NT-3, NT-4, BDNF, and BMP2/4 includes at least one protein selected from the group consisting of Wnt3a, Bcl-2, and CyclinD-1.

In a preferred embodiment, a hair catagen inducer, for example, the conditioned media of stem cells stimulated for 22 to 26 hours by adding TGF-β, and most preferably by stimulating the stem cells by about 24 hours. Preferably, the stimulated stem cells are cultured for a selected period of time of 1 to 3 days. The representative preparation method may be referred to Examples of the present invention.

Accordingly, in another exemplary embodiment, the present invention includes a method for preparing a conditioned medium of stem cells for preventing hair-loss and promoting hair-growth by stimulating stem cells with the hair catagen inducer(s).

The final concentration of the conditioned medium of stem cells is preferably about 10% to 50%, more preferably about 10% to 30%, even more preferably 20% to 30%, and most preferably about 25%.

In particular, the stem cell according to the present invention may be proliferated and cultured by a known method in the art.

As a suitable medium, any usable medium, that may be prepared in a laboratory along with appropriate components necessary or developed for culturing animal cells, and in particular mammalian cells, for example, anabolic carbon, nitrogen, and/or a trace amount of nutrients, may be used.

The medium is any basal medium suitable for the growth of animal cells, as a non-limiting example, and the basal medium generally used in culture may include Minimal Essential Medium (MEM), Dulbecco modified Eagle Medium (DMEM), Roswell Park Memorial Institute Medium (RPMI), and Keratinocyte Medium (KM), and any medium used in the art may be used without limitation. Preferably, the medium may be selected from the group consisting of α-MEM medium (GIBCO), KM (Keratinocyte medium), KBM (Keratinocyte Basal medium), EpiLife KM (Keratinocyte-EpiLife medium) medium, DMEM medium (Welgene), MCDB 131 medium (Welgene), IMEM medium (GIBCO), DMEM/F12 medium, PCM medium, M199/F12 (mixture) (GIBCO), and MSC expansion medium (Chemicon).

To the basal medium, an anabolic supply source of carbon, nitrogen, and a trace amount of nutrients, and as a non-limiting example, a serum supply source, growth factors, amino acids, antibiotics, vitamins, reducing agents, and/or a saccharide supply source may be added. However, it should be obvious that one of ordinary skill in the art may select the most appropriate medium for stem cells derived from various tissue origins or combine and appropriately culture according to a known method. In an embodiment of the present invention, α-MEM medium, KM medium, etc., were used.

Additionally, it is obvious that the stem cells may be cultured while regulating the conditions such as culturing environments, time, temperature, etc., based on the conventional knowledge in the art.

In an exemplary embodiment, mesenchymal stem cells are cultured in an α-MEM medium until cell confluence becomes about 80˜90%, preferably about 90%, and for example, wash the mesenchymal stem cells using PBS, etc., and then cultured further in KM medium for about 20 to 25 hours, and preferably for 24 hours.

As used herein, the term “confluence (%)” is a term expressing the cell concentration per area (degree of saturation) conventionally used in the art, and a unit frequently used by one of ordinary skill in the art in experiments, relatively representing the number of cells per unit area (cell concentration). The present invention also includes a method for preparing these stem cells with a size of 8 μm or less, and a conditioned medium for the same.

Meanwhile, the method may further include a step of treating the stem cells cultured in a medium according to the present invention, with trypsin.

When the cultured stem cells are treated with trypsin, stem cells in the shape of a mononuclear cell can be obtained. In particular, trypsin is treated to inhibit the agglutination between cells to thereby obtain a single cell shape, and any material which can inhibit the formation of agglutination among cells may be used.

The culture of the stem cells may be performed using a container conventionally known in the art. For example, the stem cells may be cultured using a three-dimensional bioreactor or spinner, or in a general adherent container.

The present invention relates to a use of a conditioned medium, with extremely excellent effects of preventing hair-loss and promoting hair-growth, stimulated by at least one hair catagen inducer selected from the group consisting of TGF-β, IFN-γ, FGF-5, IL-1β, TNF-α, K17, NT-3, NT-4, BDNF, and BMP2/4.

In particular, the conditioned medium of the stem cells stimulated by these factors include Wnt3a, Bcl-2, CyclinD-1, etc., which are known as signal transduction proteins associated with hair tissue differentiation, and thus they not only have the effect of simply delaying the hair-loss symptoms but also the effect of hair-growth, i.e., the effects of hair formation and growth, by the effects of formation of real dermal papilla cells, promotion of the growth in length, an increase in the number and size of follicles, increase in the thickness of scalp, etc.

Examples of drugs used to decrease DHT, which is a cause of hair-loss, which has been used so far, include Propecia® prepared using finasteride. However, the principal mechanism of the drug is that the drug inhibits the action of 5-α to reduce DHT thereby remedying hair-loss. Propecia® only has the effect of blocking 5-α reductase to inhibit DHT formation thereby maximally delaying the progress of a male-type hair-loss. Therefore, Propecia® is a therapeutic agent for males, and thus has a limitation that it is not an agent for promoting hair-growth but only an agent for preventing hair-loss.

However, the conditioned media of stem cells stimulated by at least one hair catagen inducer selected from the group consisting of TGF-β, IFN-γ, FGF-5, IL-1β, TNF-α, K17, NT-3, NT-4, BDNF, and BMP2/4, and most preferably, an inducer including TGF-β, has an advantage in that it is an agent for preventing hair-loss, and at the same time, it is an excellent agent for promoting hair-growth.

Herein below, the hair-loss prevention and hair-growth characteristics of the conditioned media of stem cells stimulated by at least one hair catagen inducer selected from the group consisting of TGF-β, IFN-γ, FGF-5, IL-1β, TNF-α, K17, NT-3, NT-4, BDNF, and BMP2/4 will be described.

(i) Reduces the conversion time from telogen phase to anagen phase during hair cycle.

Human hairs are being lost while periodically repeating anagen phase, catagen phase, and telogen phase and then formed again, and the hair cycle is established by hormonal regulation or the regulation of numerous growth factors. Dermal papilla cells have a cycle of an anagen, where growth becomes active, a catagen, where degeneration starts, and a telogen. Upon receipt of a signal from a neighboring cell, the dermal papilla cells enter into an anagen phase, and cell renewal is established, and eventually, led to formation of new hairs.

The conditioned medium of the present invention stimulated by the inducer(s) does not have a temporary effect due to hormones, etc., but has an effect of perpetual hair-growth effect by normalizing the hair cycle regulation.

(ii) Forms dermal papilla (DP) cells, promotes the growth in length, and significantly increases the number and size of follicles.

Hair follicles are accessory organs of skin possessed only by mammals, and are generated from the fetal stage and formed by the interaction between epithelium and mesenchyme.

The generation of follicles at the fetal stage is triggered by the signal from the dermis, and as a result, the epithelium becomes thick and forms a plate. The dermal signal coming out of the thick epithelial plate induces agglutination of dermal cells derived from mesenchyme, and the dermal signal again comes out of the thus formed agglutinated body. The signal promotes the proliferation of the dermal cells, and simultaneously induces the invasion into the dermis of the epithelial cells, thereby surrounding the circumference of the agglutinated body, and subsequently forming dermal papillas. As such, the first hair follicle structure is formed, and as the epithelial cells are continued to proliferate and differentiate, they become developed into matured hair follicles which form hairs. The interaction between matrix cells of hair follicles and dermal papilla cells through the basement membrane in the matured follicles causes a specified differentiation of the matrix cells of hair follicles, and as a result, forming hairs and making them grow. Additionally, the interaction generates a cycle of hair follicles, maintains organs, and determines the biological characteristics such as thickness and shape of hairs.

In the hair follicles, the two important factors that determine the biological characteristics are outer root sheath (ORS), which is a hair follicle epithelium, and mesenchyme-derived dermal papilla (DP), and hairs grow and become lost through the repetition of hair cycle.

In an embodiment of the present invention, it was confirmed that the conditioned medium of stem cells stimulated by TGF-β using hDPCs, ORS, hKC, and HaCaT cells effectively proliferated the dermal papilla (DP) cells.

Additionally, it was confirmed via animal experiments that the promotion of the length of hair follicles of primary DP cells, the promotion of hair follicle formation, the speed of hair growth, and the amount were very excellent.

(iii) Increases the thickness of the skin of scalp.

Furthermore, the conditioned medium of stem cells stimulated by the above factor(s) can also effectively increase the thickness and length of the skin, and thereby improve the overall environments associated with hair-growth.

As such, based on the novel fact that the treatment of the hair catagen inducers, in particular TGF-β, BMP, etc., although they are known as hair-loss inducing materials, on stem cells can secreted proteins which are effective for hair-growth, the present invention exhibits the effects of improving the overall environments necessary for hair-growth.

In an embodiment of the present invention, it was confirmed through in vivo experiment using mice as well as in vitro and ex vivo experiments that the conditioned medium of umbilical cord blood-derived stem cells exhibit extremely excellent hair-growth effect.

An example of in vivo experiments that can observe hair-growth possibility may be to confirm the effect of shortening the telogen phase in a mouse with a normal hair-growth cycle and rapidly inducing the anagen phase. In particular, a C57/BL6 mouse or a C3H mouse may be conventionally used because these mice enable the observation of hairs with melanin colors. Nude mice enabling the observation of proliferation of DP that enables hair-growth and proliferation of hair follicles are used. In the present invention, any mice used for the confirmation of hair-growth effect, known in the art may be used.

However, it is preferable that C3H mice are used. Unlike the general mice having about two weeks of telogen phase, the C3H mice can maintain at least 4 weeks of telogen phase, and are thus more useful as an alopecia areata mouse model. That is, the superiority of hair-growth effect may be evaluated by confirming the effect of inducing the hair cycle of the C3H mice into an anagen phase.

Accordingly, in an aspect, the present invention relates to a composition for promoting hair-growth containing, as an active ingredient, a conditioned medium of stem cells stimulated with at least one hair catagen inducer selected from the group consisting of TGF-β, IFN-γ, FGF-5, IL-1β, TNF-α, K17, NT-3, NT-4, BDNF, and BMP2/4, and most preferably an inducer including TGF-β, a method of preparing the same, and a method of preventing hair-loss and promoting hair-growth using the same.

The composition may be contained at an effective concentration of 10% to 50% (v/v) not showing any cytotoxicity, preferably 20% to 30% (v/v), and most preferably 25% (v/v), but is not limited thereto.

In an embodiment of the present invention, the composition of the present invention may include a pharmaceutical composition and/or a cosmetic composition.

Pharmaceutical Composition

In another aspect, the present invention may provide a pharmaceutical composition for promoting hair-growth containing, as an active ingredient, a conditioned medium of stem cells stimulated with at least one hair catagen inducer selected from the group consisting of TGF-β, IFN-γ, FGF-5, IL-1β, TNF-α, K17, NT-3, NT-4, BDNF, and BMP2/4, and most preferably an inducer including TGF-β.

Hair-loss is largely divided into a cicatricial hair-loss and a non-cicatricial hair-loss, and the non-cicatricial hair-loss includes a congenital hair-loss, a male-pattern hair-loss, an alopecia areata, etc. In the present invention, the hair-loss includes all of the above, and also is not limited thereto.

The pharmaceutically acceptable carriers to be contained in the pharmaceutical composition of the present invention are those conventionally used in the manufacture, and may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil, etc., but are not limited thereto. The pharmaceutical composition of the present invention may further contain a lubricant, a humectant, a sweetener, a flavoring agent, an emulsifier, a suspending agent, a preservative, etc., in addition to the above components.

The appropriate amount of the dose of the pharmaceutical composition of the present invention may vary depending on various factors including the formulation methods, administration methods, age, body weight of a patient, severity of a disease, foods, duration of administration, administration routes, release rate, and reaction sensitivity, and experienced medical practitioners can easily determine and prescribe an effective dose for the intended treatment. Meanwhile, the dose of the pharmaceutical composition of the present invention is not limited thereto, and may be 0.01 mg/kg to 2000 mg/kg (body weight) per day.

The pharmaceutical composition of the present invention may be administered orally or parenterally. When administered parenterally, the pharmaceutical composition may be administered via intravenous injection, subcutaneous injection, muscular injection, intraperitoneal injection, dermal administration. Preferably, the pharmaceutical composition is parenterally administered. Preferably, the administration route of the pharmaceutical composition of the present invention may be determined according to the type of the disease.

For example, the pharmaceutical composition of the present invention may be more preferred to be locally (topically) administered to be applied on the skin. The area for applying the pharmaceutical composition of the present invention includes not only the scalp but any part of the body that requires hair-growth. For example, an area where hairs or other body hairs are damaged due to a scar caused by an injury, or areas which require an effect of beauty care such as a wide forehead or M-type forehead, eyelashes or eyebrows, and atichia may be used for their improvement.

Preferably, the composition of the present invention may be administered via dermal administration via coating or injection, and more preferably via coating.

In particular, in the case of administering via injection, it is recommended that the composition is injected into the dermal layer while the aperture at the end of the needle of a syringe faces upward, so that the composition is delivered into capillaries after it is sufficiently diffused into the dermal layer of the skin, that is, in order to prevent the composition from being immediately entered into the dermal layer after a short retention on the dermal layer.

Cosmetic Composition

In still another aspect, the present invention relates to cosmetic composition for promoting hair-growth containing, as an active ingredient, a conditioned medium of stem cells stimulated with at least one hair catagen inducer selected from the group consisting of TGF-β, IFN-γ, FGF-5, IL-1β, TNF-α, K17, NT-3, NT-4, BDNF, and BMP2/4, and most preferably an inducer including TGF-β.

The cosmetic composition of the present invention may be prepared in any formulation conventionally manufactured in the art. For example, the cosmetic composition may be formulated into latex, cream, cosmetic water, a pack, a foundation, a lotion, a beauty liquid, hair cosmetics, but is not limited thereto.

Preferably, the cosmetic composition may be prepared as a composition, by adding a conventional additive, such as a shampoo, a hair rinse, a hair tonic, a hair gel, a hair lotion, a hair pack, a hair spray, a hair moose, a hair treatment, a hair dye, a hair conditioner, a mixed type thereof for promoting hair-growth, e.g., a mixed type between a shampoo and a rinse, a mixed type between a rinse and a treatment, a liquid agent for hair-growth, etc., and an aerosol type thereof may be included.

When the formulation type is a paste, cream, or gel, the component for a carrier may be an animal oil, a vegetable oil, wax, paraffin, starch, tragacanth, a cellulose derivative, polyethylene glycol, silicone, bentonite, silica, talc, or zinc oxide, etc. When the formulation type is powder or spray, the component for a carrier may be lactose, talc, silica, aluminum hydroxide, calcium silicate or polyamide powder, and in particular, in the case of spray, a propellent such as chlorofluoro hydrocarbon, propane/butane or dimethyl ether may be included. When the formulation type is a liquid or an emulsion, the component for a carrier may be a solvent, a solubilizing agent, or an emulsifying agent, e.g., water, ethanol, isopropanol, ethyl carbonate, ethyl acetate, benzyl alcohol, benzyl benzoate, propylene glycol, 1,3-butylglycol oil, glycerol aliphatic ester, polyethylene glycol or adiposety ester of sorbitan. When the formulation type is a suspension, the component for a carrier may be a liquid phase diluent such as water, ethanol or propylene glycol, ethoxylated isostearyl alcohol, a suspending agent such as polyoxyethylene sorbitol ester and polyoxyethylene sorbitan ester, microcrystalline cellulose, aluminum metahydroxide, bentonite, agar or tragacanth, etc. When the formulation type is a surfactant-containing cleansing, the component for a carrier may be aliphatic alcohol suladiposee, aliphatic alcohol ether suladiposee, sulfosuccinate monoester, isethionate, an imidazolinium derivative, methyl taurate, sarcosinate, adiposety acid amide ether suladiposee, alkylamidobetaine, aliphatic alcohol, adiposety acid glyceride, adiposety acid diethanolamide, a vegetable oil, a lanolin derivative or ethoxylated glycerol adiposety acid ester, etc.

The components to be contained in the cosmetic composition of the present invention may include components conventionally used in the cosmetic composition, in addition to the active ingredients, e.g., antioxidants, stabilizers, solubilizing agents, vitamins, conventional adjuvants such as pigments and flavoring agents, and carriers.

The cosmetic composition may be prepared by any method conventionally used.

Preferably, the cosmetic composition for preventing hair-loss and promoting hair-growth may be used via dermal application by directly applying it on scalp or hair or injecting it thereon.

The amount of application of the mixed extract as an active ingredient contained in the composition of the present invention, based on an adult, is 40 mg/kg or less, and preferably 20 mg/kg to 40 mg/kg.

The method of applying the composition on the skin may include any method disclosed in the art. The cosmetic composition of the present invention may be used after a single application or double-application, or may be used after a double-application with a cosmetic composition other than that of the present invention. Additionally, the cosmetic composition of the present invention with excellent effects of skin protection may be used according to a conventional method of use, and the frequency of its application may vary according to the skin condition or taste of a user.

Method of Hair-Loss Treatment

Additionally, in another aspect, the present invention relates to a method for treating hair-loss by promoting hair-growth using a composition for promoting hair-growth containing, as an active ingredient, a conditioned medium of stem cells stimulated with at least one hair catagen inducer selected from the group consisting of TGF-β, IFN-γ, FGF-5, TNF-α, K17, NT-3, NT-4, BDNF, and BMP2/4, and most preferably an inducer including TGF-β, a method of preparing the same, and a method of preventing hair-loss and promoting hair-growth using the same.

In the above method of treating hair-loss, the details of the conditioned medium of stem cells or the composition are the same as described above.

In the above method of treating hair-loss, in particular, it is preferable to use a dermal administration method using a coating or an injection method. In particular, it is preferable that the active ingredient of the composition is injected into the dermal layer while the aperture at the end of the needle of a syringe faces upward, so that the composition is delivered into capillaries after it is sufficiently diffused into the dermal layer of the skin, that is, in order to prevent the composition from being immediately entered into the dermal layer after a short retention on the dermal layer.

As described above, the function of the conditioned medium of stem cells stimulated by the hair catagen inducer, explained previously, was explained with reference to the pharmaceutical composition and the cosmetic composition. However, it should be obvious to one of ordinary skill in the art to which the present invention pertains that the present invention relates to various forms of compositions for preventing hair-loss and promoting hair-growth containing, as an active ingredient, the conditioned medium of stem cells stimulated by a hair catagen inducer, and a method of using the compositions with various applications.

### DETAILED DESCRIPTION OF THE INVENTION

Hereinbelow, the present invention will be described in detail with reference to accompanying exemplary embodiments. These embodiments are disclosed for illustrative purposes only and it should be obvious to those skilled in the art that they should not be construed as limiting the scope of the present invention.

Although the present inventors used umbilical cord blood-derived stem cell and TGF-β, it should be obvious to those skilled in the art that other source-derived stem cells and other hair catagen inducers (Current Biology 19, R132-R142, Feb. 10, 2009; J Invest Dermatol 124:675-685, 2005) may be used as well.

Materials and Methods

1. Preparation of a Conditioned Medium of Stem Cells

(1) Isolation and Culture of Stem Cells

In the present invention, human umbilical cord blood-derived mesenchymal stem cells provided by Medipost Co., Ltd. (Korea) were used. The cells can be obtained from the step of collecting the umbilical cord blood, and the step of isolating the mesenchymal stem cells from the umbilical cord blood and culturing the same. The details of each step are described herein below.

In the step of collecting umbilical cord blood, in the case of normal spontaneous vaginal delivery (NSVD), after delivery of a baby, the umbilical cord blood is collected from the expelled umbilical cord vein while the placenta still remains in the uterus, or in the case of a cesarean section, the umbilical cord blood is collected from the umbilical cord vein in a state that the placenta is also expelled out of the uterus after the delivery of a baby.

In the present invention, when umbilical cord blood is collected from the umbilical cord vein expelled out of the uterus after delivery, umbilical cord blood is collected via aseptic operation after the birth of a newborn baby from the umbilical cord vein which had been connected to the placenta and a fetus. Once the umbilical cord vein is acquired, umbilical cord blood is collected into an umbilical cord blood collection bag (pouch) containing an anticoagulant using a collecting needle. Regarding the method of isolating stem cell from the thus collected umbilical cord blood and culturing the same, any conventional method including that disclosed in Korean Patent No. 10-0494265 may be used (Pittinger M F, Mackay A M, et al., Science, 284: 143-7, 1999; Lazarus H M, Haynesworth S E, et al., Bone Marrow Transplant, 16: 557-64, 1995).

The present inventors separated mononuclear cells from the thus collected umbilical cord blood by centrifugation, washed a few times to remove impurities, and planted the mononuclear cells into a culturing container at an appropriate concentration and cultured. Here, the cells, when observed under a phase contrast microscope, proliferating in the form of a colony with a long homogeneous spindle shape were confirmed to be the mesenchymal stem cells. When the cells are grown to be confluent the cells were then subcultured and proliferated until an appropriate number of cells were obtained.

(2) Preparation of Sample Conditioned Media

Sample conditioned media were prepared from hUCB-MSC. In an incubator kept at 37° C. and 5% CO₂, the cells under storage (stored in an LN2 tank) were thawed and cultured, and in particular, the cells were cultured until cell confluence reached about 90% in an α-MEM (GIBCO) medium containing FBS.

Then, the cells were washed 3 times with phosphate buffered saline (PBS), cultured in a Keratinocyte Medium (KM) not added with phenol red for 24 hours, and the culture medium was collected, and the entire process was repeated for 3 days. Then, the collected media were filtered (Top Filer System, Nunc), respectively, and stored in being refrigerated and frozen to be used.

(3) Preparation of Stimulated CM

The method of CM preparation is almost the same as the method described previously, but when culturing the umbilical cord blood-derived stem cells were cultured, the cells were treated with TGF-β (10 ng/ml) in a medium not containing phenol red, and stimulate the stem cells for 24 hours.

The medium treated with the TGF-β was washed 3 times with PBS, and replaced with a fresh KM medium not containing phenol red, repeated the 24 hour culturing for 3 days and collected. The thus collected conditioned media treated with TGF-β were filtered, respectively, and, for experimental use, diluted in a KM medium not containing phenol red to a final concentration of 10%, 25%, and 50% to be used.

2. Preparation of C3H Mice for Observation of Hair-Growth

The site for experiment was Gyeongi Biocenter (IACUC Project No: IACUC2014-4-10) of Medipost ORB Approval No: 131021-1), and C3H mice were purchased from Saeronbio Inc. (Korea) and prepared in Jackson lab.

In particular, the C3H mice (Jackson lab, Japan) used in the experiment of the present invention start telegen phase when their hairs are removed. Unlike other species, the C3H mice require an extremely long time for their conversion into the anagen phase, and thus they are used as an animal model for studying the hair-growth effect. That is, unlike other mouse species, they have very long telogen phase without treatment of an inducer drug, and thus they are an excellent animal model for confirming the effect of hair-growth (Journal of Investigative Dermatology (2005) 124, 288-289).

The color of C3H mice in their skin surface turn to black during the anagen phase, which is a hair-growth period, whereas their skin surface turn to pink during the catagen phase, and thus it is possible to confirm the period of hair-growth by observing their skin color.

The present inventors obtained 7-week old C3H mice, and allowed them to adapt to a new environment through a 1 week adaptation period. Additionally, they inoculated the mice with the conditioned media of mesenchymal stem cells derived from umbilical cord blood to observe the telogen phase (catagen phase) of hair cycle and the conversion time into the anagen phase. In this example, the mouse injected with PBS was used as a negative control.

Meanwhile, the mice were anesthetized for hair removal. An anesthetic, in the amount of 15.83 ml was prepared by mixing 3.36 ml of Rompun (Bayer Korea Ltd., 2094L, Korea) with 5 ml of zoletil (Bar code #3UHC, Korea) and adding 7.47 ml of saline (JW-Pharma, REG#10055, Korea), and anesthetized the mice using 20 μl of the same using an insulin syringe (BD Ultra-Fine™ II, Korea).

Then, after confirming that the mice were anesthetized, the mice hairs were removed using a hair trimmer. The mice were place on a clean paper, and the primary hair removal was performed in a direction opposite to the hair-growth direction, left the mice thereat for 24 hours, observed the presence of any remaining hairs, and then the remaining hair was removed.

3. Topical Application of the Conditioned Medium of Stem Cells of the Present Invention

As a topical method, the conditioned medium of the present invention was applied in an amount of 100 μl at 12 hour intervals onto the outer layer of the mice skin, and scrubbed 8 times in the same direction so that they could be smeared into the skin while carefully attending not to stain the neighboring region. The conditioned medium was applied at point 4 in an amount of 100 μl, and the amount of hair-growth, hair thickness, the location of color, etc., were observed daily by the naked eye.

4. MTT Analysis

In order to test the toxicity and proliferation of cells, the cells were cultured in the respective medium in an incubator kept at 37° C., 5% CO₂ for 24 hours, placed in starvation for 24 hours, and cultured under the respective experimental condition for 24, 48, 72, and 96 hours.

Additionally, the completed respective experimental group was subjected to measurements regarding cytotoxicity and cell proliferation rate via MIT analysis. Experiments were repeated at least 3 times for securing reliability on the experiments.

Each respective experimental group, which was completed with the culturing for the establishment of MIT analysis experimental method, was treated with 5 mg/ml MTT reagent to a final concentration of 1 mg/ml, cultured for 4 additional hours. Then, the supernatant was discarded, and the resultant was dissolved in DMSO, and the resulting solution was transferred into a 96-well plate in an amount of 200 μl/well, and the absorbance was measured at 570 nm by ELISA reader.

5. Separation of Primary hDPCs and Primary ORS as Primary Cells

The autopsy tissue of the occipital region was prepared in saline, and separated into a one follicular unit using a blade to cut the bulb, and the hair shaft was removed. One of the two syringes fixed the lower portion (the lower portion of DP) of the hair bulb while the other syringe was used to slightly touch the upper portion (the higher portion of DP) of the hair bulb so that the dermal papilla (DP) could come out.

The dermal papilla cells were placed at the end of the syringe, and a mixture (DMEM+20% FBS+1% antibiotics+1% Fungizone), and about 10 dermal papilla cells were added into a 35 mm type I collagen coating dish, and cultured for 10 days while adding the amount in deficiency without replacing of the medium. After confirming that the dermal papilla cells were adhered to the dish, the medium was replaced once in 3 days, and subcultured for use either when the cell confluence was reached or when it became the 4^{th} week.

6. Patch Assay

When the C57BL/6 mice gave birth their skins were peeled off, washed off the blood stains, sterilized with povidine, and washed again with saline to remove povidine.

After removing adipose layer from the peeled-off skin, the mice were treated with collagen/dispase, and cultured overnight at 4° C. The dermis and epidermis were separated using a forcep, vortexed for 15 minutes, sieved the tissues with a strainer, and separated (cell down) by centrifugation. The number of cells was counted, divided into 1*10̂6, added with 100 μl of the culture medium, respectively, and resuspended to release the cells. An insulin syringe was used for subcutaneous injection on 4 parts on the back of a nude mouse. Two weeks after the injection, the skins were autopsied and the number of hair follicles formed inside was confirmed.

7. Culturing of Primary Hair Organ

Hair organs were collected by an autopsy on the occipital region, and prepared in saline, trimmed with a hair follicle unit, and cut up to the point immediately below the sebaceous gland. The medium was replaced once in 3 days and the length was measured.

### Example 1

### Confirmation of Hair-Growth Effect of Umbilical Cord Blood-Derived Mesenchymal Stem Cells (hUCB-MSC) Medium

Since the initiation of hair-growth is possible during the formation of dermal papillar (DP) and proliferation, the possibility of hair-growth was examined by observing the features of increasing the DP growth by hUCB-MSC-CM.

The evaluation on the effect of hair-growth by the conditioned media of stem cells stimulated by TGF-β and the unstimulated conditioned medium was performed so that the final concentrations of the conditioned media became 10%, 25%, and 50%, and they were used for in vivo and ex vivo experiments.

1-1 In Vitro Experiment

The effect of the condition media were examined by in vitro experiment using the primary human dermal papilla cells (DPCs).

First, in order to test the toxicity and proliferation of cells, the cells were cultured in the respective medium in an incubator kept at 37° C., 5% CO₂ for 24 hours, placed in starvation for 24 hours, and cultured under the respective experimental condition for 24, 48, 72, and 96 hours, and the toxicity and proliferation of cells were measured via MIT analysis.

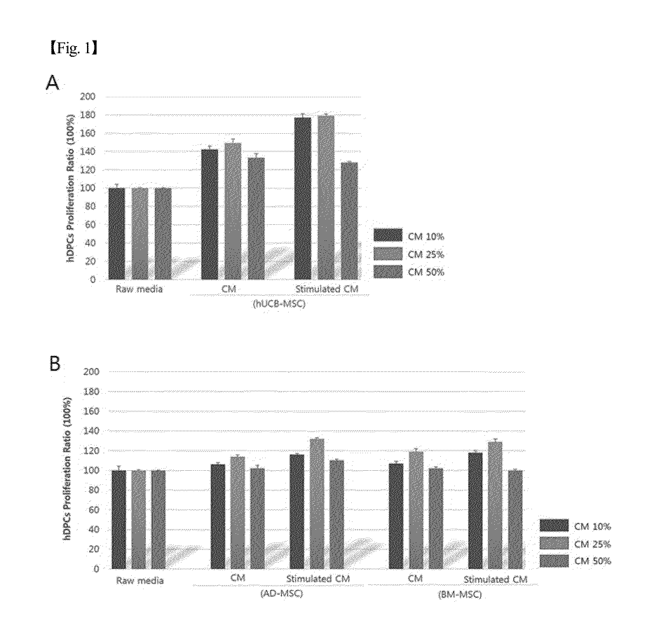

As a result, as shown in FIG. 1, the conditioned medium of umbilical cord blood-derived stem cells stimulated by TGF-β showed the highest cell viability.

Furthermore, the proliferations of AD-MSC and BM-MSC were also measured (FIG. 1 and Table 1).

TABLE 1

| | CM | | | stimulated CM | | |
|---|---|---|---|---|---|---|
| UCB | 142 | 149 | 133 | 177 | 178.9 | 128 |
| AD | 106 | 114 | 102 | 116 | 132 | 110 |
| BM | 107 | 119 | 102 | 118 | 129 | 100 |

From these results, we can confirmed that the stem cells stimulated by TGF-β have a superior proliferation to non-stimulated media

1-2 Western Blotting

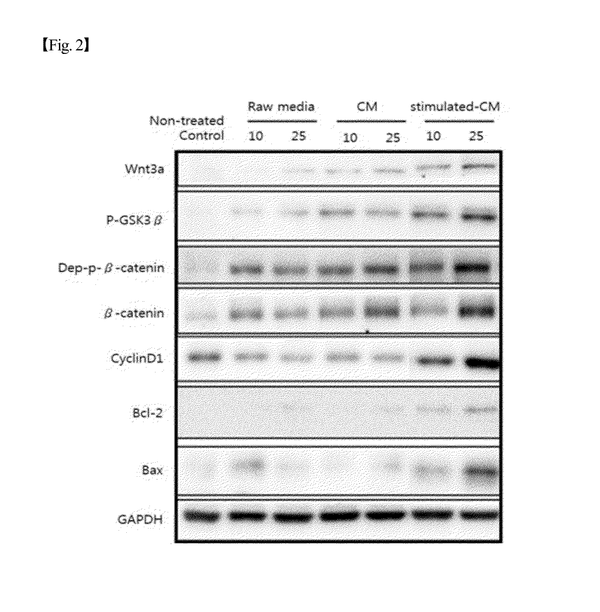

Meanwhile, in order to confirm the signal transduction system associated with hair tissue differentiation by the conditioned media of stem cells stimulated by TGF-β, the hDPCs were treated with the conditioned media candidates, and the level of proteins associated with hair tissue differentiation was confirmed via western blot, and the results are shown in FIG. 2.

As a result, the conditioned media of stem cells stimulated by TGF-β showed high expression of Wnt3a, Bcl-2, CyclinD-1, etc., which are known as signal transduction proteins associated with hair tissue differentiation. These results reveal that the TGF-β of the present invention has an effect on the stem cells regarding hair tissue differentiation.

That is, the umbilical cord blood-derived stem cells of the present invention stimulated by TGF-β effectively secrete Wnt3a, Bcl-2, CyclinD-1, etc., which are the signal transduction proteins associated with hair tissue differentiation, and thus the conditioned media containing the same exhibit the most superior hair-growth effect according to hair growth (FIG. 2).

1-3 Ex Vivo Experiment

The present inventors additionally performed ex vivo experiments on hair-growth effect by culturing the primary hair organ.

To this end, as an ex vivo model enabling the observation on the possibility of hair-growth, the human primary dermal papilla (DP) from hair follicles collected from the human scalp tissues was prepared and cultured in an experimental plate. The promotion of growth in length of DP according to the material for treatment was compared by observation. That is, the growth in the primary DP length was observed by treating the experimental group and the control group, and the effective CM was selected, or the effect of CM on hair-growth was compared by observation.

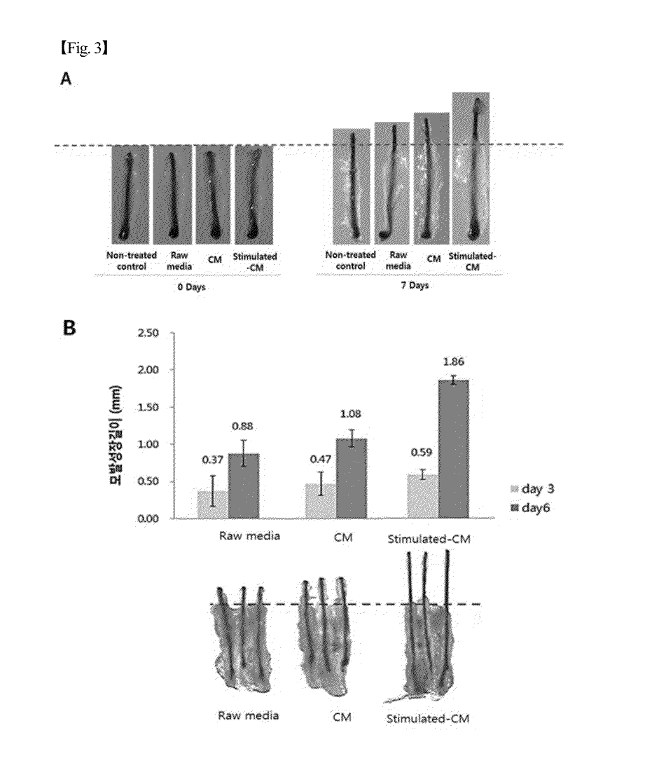

As a result, as shown in FIG. 3, both the non-stimulated CM, and CM stimulated with TGF-β induced hair-proliferation, compared with the untreated control group, and in particular, the CM stimulated by TGF-β showed a higher hair-growth effect than non-stimulated CM (FIG. 3A).

Additionally, when the growth length of hair follicles in the four repeated experiments, the conditioned media stimulated by TGF-β showed a higher average growth length of hair follicles compared to the non-stimulated conditioned medium (FIG. 3B).

### Example 2

### Patch Assay

The effect of the conditioned media priming on umbilical cord stem cells on hair follicle formation was examined via patch assay model.

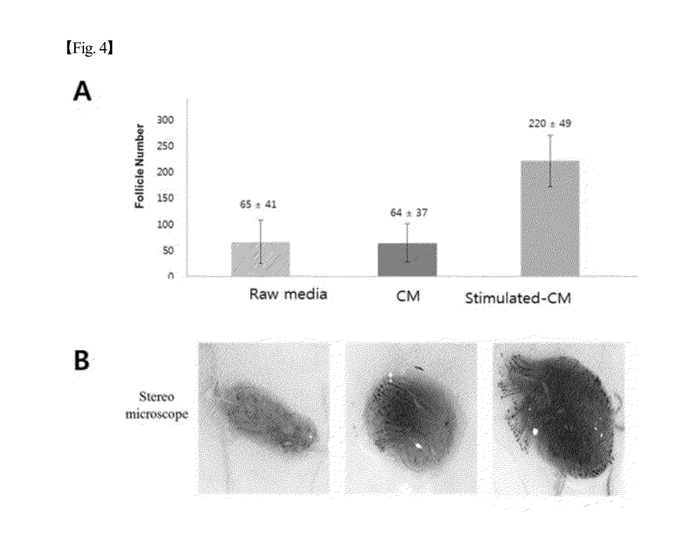

As a result of ex vivo experiment via patch assay model, the conditioned media of stem cells stimulated by TGF-β showed a significantly higher effect of forming primary hair follicles, compared to the control medium or a group treated with unstimulated conditioned medium. In particular, the conditioned media of stem cells stimulated by TGF-β showed a higher number of primary hair follicles (FIG. 4A).

Also, in an observation through a stereoscopic microscope, the conditioned media stimulated by TGF-β showed a significantly higher effect of forming primary hair follicles, compared to the control medium or a group treated with unstimulated conditioned medium (FIG. 4B).

### Example 3

### Topical Application on Mice

The effect of hair-growth was evaluated by topically applying the conditioned medium of the present invention on a C3H/HeJ mouse at telogen phase.

For the evaluation of the effects of preventing hair-loss and promoting hair-growth, animal experiments were compared and evaluated using the conditioned media stimulated by various factors. The conditioned media were applied at four locations on the back of each mouse, twice a day, and the hair-growth feature was examined in about 3 weeks.

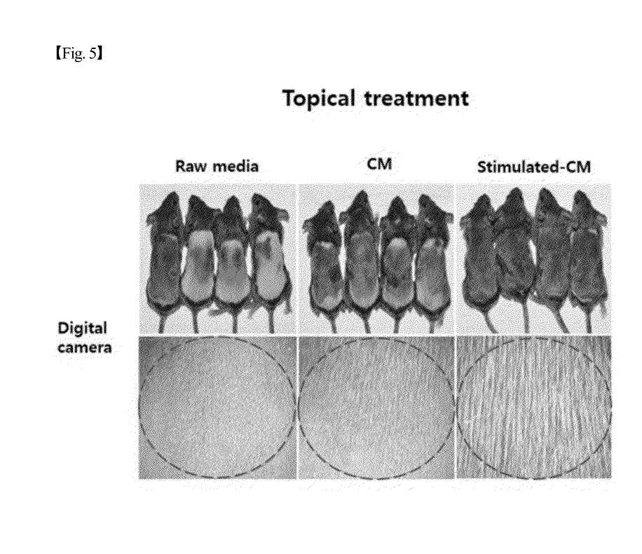

As a result, it was confirmed that the conditioned media of stem cells stimulated by TGF-β showed a higher hair-growth effect, compared to the control medium or a group treated with unstimulated conditioned medium, and among them, the conditioned media of stem cells stimulated by TGF-β was shown to have the most superior effect in various fields of hair-growth such as the speed of hair-growth, the amount of hair growth, the thickness of hair, etc., even comparing with the study results that have been reported (FIG. 5).

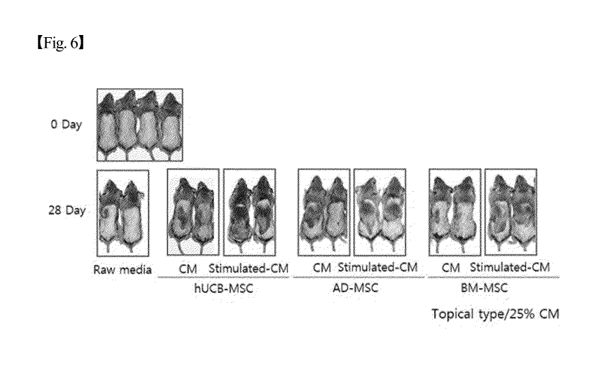

Furthermore, the hair-growth feature of C3H/HeJ mouse examined in about 28 days after treating CM and CM stimulated by TGF-β with AD-MSC and BM-MSC, the mouse in which the CM stimulated by TGF-β was applied topically, showed the superior hair-growth effect, too (FIG. 6)

As such, it can be confirmed via in vivo model that the hair-growth effect of the conditioned media of stem cells of the present invention stimulated by TGF-β is very superior in the aspects of hair-growth rage, amount, thickness, etc.

From these results, it was confirmed that the treatment of umbilical cord blood-derived stem cells with TGF-β, which has been known as a hair-loss inducing material, can surprisingly secrete proteins which are effective for hair-growth.

In particular, the conditioned media of umbilical cord blood-derived mesenchymal stem cells stimulated with TGF-β of the present invention exhibit far superior hair-growth effect than the existing stem cell-based materials, and are thus expected to be very usefully used for the prevention of hair-loss and promotion of hair-growth.

### INDUSTRIAL APPLICABILITY

The conditioned media of stem cells stimulated by the hair catagen inducer including TGF-β contain Wnt3a, Bcl-2, CyclinD-1, etc., which are known as signal transduction proteins associated with hair tissue differentiation. Accordingly, the conditioned media of stem cells shorten the conversion time from telogen phase to anagen phase during hair cycle, promote the dermal papilla cells, growth in length, increase the number and size of hair follicles, and the thickness of scalp, thereby exhibiting excellent preventing of hair-loss and promotion of hair-growth, and are thus expected to be very useful in the field that can utilize the same.

1. A composition for preventing hair-loss and promoting hair growth comprising, as an active ingredient, a conditioned medium of stem cells stimulated by at least one hair catagen inducer selected from the group consisting of TGF-β, IFN-γ, FGF-5, IL-1β, TNF-α, K17, NT-3, NT-4, BDNF, and BMP2/4.

2. The composition of claim 1, wherein the hair catagen inducer is at least one factor including TGF-β.

3. The composition of claim 1, wherein the stem cell is at least one selected from the group consisting of human tissue adult stem cells and embryonic stem cells derived from bone marrow, umbilical cord blood, adipose, blood, liver and intestines, skin, gastrointestinal tract, placenta, nerves, adrenal gland, epithelium, and uterus.

4. The composition of claim 3, wherein the stem cell is derived from umbilical cord blood, bone marrow or adipose.

5. The composition of claim 3, wherein the stem cell is derived from umbilical cord blood.

6. The composition of claim 1, wherein the conditioned media stimulated by the hair catagen inducer is obtained by stimulating the stem cells for 22 to 26 hours after adding the hair catagen inducer to the stem cells, followed by culturing for a selected period of time of from 1 to 3 days.

7. The composition of claim 1, wherein the conditioned media stimulated by the hair catagen inducer comprises at least one protein selected from the group consisting of Wnt3a, Bcl-2, and CyclinD-1.

8. The composition of claim 1, wherein the conditioned media stimulated by the hair catagen inducer has a final concentration of 10% to 30%.

9. The composition of claim 1, wherein the conditioned media stimulated by the hair catagen inducer possesses the following functions:
(i) reducing the conversion time from telogen phase to anagen phase during hair cycle;
(ii) producing dermal papilla cells and promoting the growth in length;
(iii) increasing the number and size of follicles; and
(iv) increasing the thickness of scalp skin.

10. The composition of claim 1, wherein the composition is a pharmaceutical composition or a cosmetic composition.

11. A method of manufacturing conditioned media of stem cells for preventing hair-loss and promoting hair growth by stimulating the stem cells with at least one hair catagen inducer selected from the group consisting of TGF-β, IFN-γ, FGF-5, IL-1β, TNF-α, K17, NT-3, NT-4, BDNF, and BMP2/4.

12. The method of claim 11, wherein the hair catagen inducer includes TGF-β.

13. The method of claim 11, wherein the stem cell is derived from umbilical cord blood, bone marrow or adipose.

14. A method of using a composition for preventing hair-loss and promoting hair growth comprising orally administering the composition of claim 1 by topical spreading or injection.

15. A method of treating hair-loss using conditioned media of stem cells stimulated by at least one hair catagen inducer selected from the group consisting of Wnt3a, Bcl-2, and CyclinD-1.

16. The method of claim 15, wherein the hair catagen inducer is at least one factor including TGF-β.

17. The method of claim 15, wherein the stem cell is derived from umbilical cord blood, bone marrow or adipose.

18. The method of claim 15, wherein the conditioned media of stem cells stimulated comprises at least one protein selected from the group consisting of Wnt3a, Bcl-2, and CyclinD-1.

19. A method of using a composition for preventing hair-loss and promoting hair growth comprising orally administering the composition of claim 2 by topical spreading or injection.
